(19) United States
(12) Patent Application Publication
(10) Pub. No.: US 2012/0231455 A1
(43) Pub. Date: Sep. 13, 2012

(54) PEPTIDE NUCLEIC ACID PROBES, KIT AND METHOD FOR DETECTING HELICOBACTER PYLORI AND/OR CLARITHROMYCIN RESISTANCE PROFILE AND APPLICATIONS

(75) Inventors: Nuno Filipe Ribeiro Pinto De Olivera Azevedo, Braga (PT); Laura Isabel Maciera Cerqueira, Braga (PT); Nuno Ricardo Torres Faria, Vila Verde (PT); Maria João Lopes Da Costa Vieira, Braga (PT)

(73) Assignee: Universidade Do Minho, Braga (PT)

(21) Appl. No.: 13/395,618

(22) PCT Filed: Sep. 13, 2010
(86) PCT No.: PCT/IB2010/054108

§ 371 Date: May 22, 2012

(30) Foreign Application Priority Data
Sep. 11, 2009 (PT) 104744

(51) Int. Cl. C12Q 1/68; G01N 21/64; C07K 14/00; C12N 9/96

(52) U.S. Cl. 435/6.11; 435/188; 530/358

(57) ABSTRACT

Four peptide nucleic acid probes (PNA) are for the detection of Helicobacter pylori and/or response analysis of Helicobacter pylori strains to clarithromycin. The probes are based in molecular biology methods, namely fluorescence in situ hybridization (FISH) that are used in H. pylori clarithromycin susceptibility diagnosis in several sample types, including clinical isolates and biopsies. Due to physical and chemical characteristics inherent in its structure, the probes allow a faster and more sensitive hybridization. A kit may use one or several probes described here and the related process of detection or quantification. The kit identifies H. pylori and its response to clarithromycin in clinical samples.

### FIELD OF INVENTION

The present invention refers to the field of detection process and antibiotic resistance profile of clinical relevant microorganisms. Four peptide nucleic acid probes (PNA) were developed to detect H. pylori and/or strains response to clarithromycin presence (resistance/susceptibility).

Another aspect of the present invention relates to the application of these probes and the referred detection process to a kit for detection of clarithromycin resistance of H. pylori in biological samples and therefore having clinical application.

### BACKGROUND OF THE INVENTION

H. pylori is a pathogenic bacterium that colonizes the human gastric mucosa, being associated with gastric infections such as chronic gastritis, peptic ulcers, atrophic gastritis and gastric cancer.

H. pylori infection diagnosis can be based in invasive methods, such as gastric biopsies (endoscopic exam), or non-invasive methods such as urea breath test, serologic test or stool antigen tests. The later ones are more frequently used in clinical practice because they are less expensive and more convenient to the patient.

The most commonly used tests/kits can detect a specific H. pylori enzyme, urease (US200306856 and EP0920531). However, the invasive methods are still preferably used in a more detailed diagnosis due the lack of sensitivity/specificity present in the standard methods.

In fact, with an endoscopic biopsy it is possible to obtain more reliable information concerning H. pylori presence, either using culturing based tests such as the E-test and agar dilution method or through molecular methods based in PCR (Polymerase Chain reaction). However, the first ones are fastidious and time consuming and the second ones need some extra care to avoid possible DNA contaminations that can lead to false positives or false negatives.

More recently, Peptide nucleic acid probes (PNA) have been developed and optimized for bacterial detection. The PNA molecules are DNA mimics, in which its negative charged sugar-phosphate backbone structure is replaced by an aquiral and electrically neutral, formed by repetitive (2-aminoetil)glycin units.

Although, PNA molecules lack pentoses, specific hybridization between PNA and complementary DNA sequences can occur by hydrogen bounding, obeying the Watson-Crick rules.

The electrically neutral nature of PNA is responsible for a more robust hybridization between PNA and the target sequence (rRNA and double stranded DNA) compared to typical DNA probes, traditionally used. Due to its high affinity, PNA probes have nucleotide sequences relatively shorter (8-nucleotides). A DNA probe usually has at least 18 nucleotides, due to its weak stability and lower melting temperature (Tm, requiring additional fixation and permeabilization procedures with enzymes and other agents. PNA molecules present higher resistance to proteases and nucleases, than DNA molecules.

PNA probes can be detected by microscopy or flow cytometry, by fluorescent in situ hybridization technique (FISH) when attached to a fluorochrome.

Comparing with traditional culturing methods, this technique originates more prompt results in clinical samples, also with improved efficacy, sensibility and specificity. It presents a wide range of applications and can be applied in different microbiology areas, such as microorganism's detection in human, environmental and food samples.

Some examples of probes for the detection of clinical microorganism, such as Salmonella, Bacillus anthracis, species of Staphylococcus non-aureus, human Papillomavirus, and Candida genus, that were already designed and published/patented, show the increasing interest for this technique.

A PNA probe for the specific detection of H. pylori is already available (Azevedo N. F. et al., 2003). Moreover, patent for another probe was requested (WO2008155742-A2; PT103767-A1). The latter showed quite high specificity and sensibility, overcoming the standard methods that are being used (Guimarães N. et al., 2007). Although H. pylori detection is important, it is desirable to identify the H. pylori antibiotic resistances, which is not possible using only the probes mentioned above.

In fact, the most commonly used treatment for H. pylori eradication is the triple therapy, which comprises a proton pump inhibitor and two antibiotics (eg. clarithromycin, metrodinazole, amoxicillin) for 7 to 14 days. This therapy can cause discomfort to the patient, due to antibiotic over usage and to the secondary effects that they can cause. Additionally, the success of this therapy has been hindered by an increase of H. pylori resistance to clarithromycin, which is the antibiotic of excellence used in these case. At present, clarithromycin resistance is detected by culturing methods or PCR, but these methods have the same limitations as those used for H. pylori detection and described above. Therefore, due to lack of prompt, reliable and practical methods the clarithromycin resistance of each strain of H. pylori is usually obtained after the beginning of the treatment or after its inefficiency has been proven. The present invention describes a reliable, practical and robust application of resistant strains identification and presents a significant contribution for adequate treatment choice, saving nuisance, risks, treatment time, and costs for the patient.

The FISH technique is as such an alternative method for detecting clarithromycin resistance in H. pylori. In fact, there are already some studies with DNA probes that detect H. pylori clarithromycin resistance (Trebesius et al., 2000). The clarithromycin resistance is associated with point mutations in peptidiltransferase region of the domain V of 23S rRNA of H. pylori. The most frequent point mutation referred in studies is the A2143G followed by A2142G and A2142C. In one study, data from several countries was compiled and it was calculated that these three point mutations presents an associated prevalence with clarithromycin resistance of 84%(Megraud, 2004).

### SUMMARY OF THE INVENTION

The present invention refers to a peptide nucleic acid (PNA) probe or sets of PNA probes for the detection of Helicobacter pylori (identification/quantification) and/or its response to clarithromycin (resistance/susceptibility). Such probes can recognize the microorganism 23S rRNA or the genomic sequences corresponding to the mentioned rRNA.

PNA probes have physiochemical characteristics that are inherent to its structure and when they are applied in a FISH-based method, allow a more prompt, robust and specific analysis compared to DNA probes. This factor can be extremely important since the difference between H. pylori resistant and susceptible strains can be of one base.

One of the problems solved by the present invention is the detection/quantification of H. pylori and/or clarithromycin response (susceptibility or resistance). It provides a reliable and prompt method for determining H. pylori presence within a biological sample, and quantification and determination of their response to clarithromycin in order to be able to determine the most convenient treatment in a quick and safe way. Another aspect of the present invention is related to the development of a kit based on the application of these probes to fluorescence in situ hybridization (FISH), allowing the detection of clarithromycin resistance of H. pylori in biological samples in a prompt and easy way. The probes can be used in multiplex or separately.

The present invention describes a set of PNA probes that identify/measure H. pylori presence or detect the response of H. pylori to clarithromycin, or detect H. pylori and its response to clarithromycin, which comprises at least one of the nucleotide sequences being at least 86% structurally similar to SEQ ID Nos. 1-4.

In a preferred embodiment of the present invention, the PNA probes here described detect the target sequence in rRNA, in rDNA or in complementary sequences of rRNA of Helicobacter pylori.

In a further preferred embodiment of the present invention the PNA probe comprises the following sequences:
At least one of the nucleotide sequences, at least 86% structurally similar to SEQ ID Nos. 1-3, detects H. pylori and its resistance to clarithromycin;
At least one of the nucleotide sequences, at least 86% structurally similar to SEQ ID Nos. 1-4, detects H. pylori and its susceptibility to clarithromycin.

In a more preferable embodiment of the present invention, the sequences are linked to at least one type of fraction detectable. Being that the type of fraction detectable used may be selected from one of the following groups: a conjugate, a branched detection system, a chromophore, a fluorophore, radioisotope, an enzyme, a hapten or a luminescent compound, among others.

In an even more preferable embodiment of the present invention, the fluorophore group can be at least one of the following: Alexa series fluorophores, Alexa fluor series, cyanin, 5 - (and -6) Carboxy-2′,7′-dichlorofluorescein, 5-ROX (5-carboxy-X-rhodamine, triethylammonium salt), among others.

It is still the subject of the present invention a kit for H. pylori detection or, H. pylori clarithromycin response detection or, the detection of H. pylori and its response to clarithromycin in biological samples, which comprises at least one the probes earlier described.

In a more preferable embodiment of the present invention, the kit may still present at least one of the following solutions: a fixation solution, a hybridization solution and a washing solution.

In yet another preferred embodiment of the present invention, the fixation solution can comprise paraformaldehyde and ethanol namely 2-8% (wt/vol) of paraformaldehyde and 25-75% (vol/vol) of ethanol and/or hybridization solution may have formamide.

It is still the subject of the present invention a method for H. pylori detection, or for H. pylori clarithromycin response or for the detection of H. pylori and its response to clarithromycin in biological samples, which uses at least one of the PNA probes earlier described and comprises the following steps:
The contact of the PNA probes with biological samples;
PNA probes hybridization with the target sequence of the microorganisms within the biological samples;
The hybridization detection as an indication of detection and quantification in biological samples.

The biological samples can be derived from blood, air, food, water, biopsies, among others.

In a further embodiment of the present invention, the hybridization may be done by fluorescence.

It is still the subject of the present invention the use of the PNA probes earlier described, the use of the kit earlier described and of the methodology of the detection of H. pylori or detection of H. pylori response to clarithromycin, or the detection of H. pylori and its response to clarithromycin in biological samples.

### GENERAL DESCRIPTION OF THE INVENTION

The present invention comprises PNA probes, reagents, methods and a kit intended for the detection and quantification of Helicobacter pylori strains or, detection and quantification of Helicobacter pylori response to clarithromycin or, detection and quantification of Helicobacter pylori and its response (resistance and susceptibility) to clarithromycin. The highest specificity of the PNA probes (in relation to DNA probes) will permit a better discrimination of nucleotide sequences related with one or two different nucleotides. In the present invention this aspect assumes particular relevance since the difference between H. pylori clarithromycin susceptible and resistant strains is precisely of one base.

The PNA probes described in the present invention are able to detect rRNA, genomic sequences corresponding to rRNA (rDNA), or its complementary sequences, allowing the specific detection of the target species and even determine the response to antibiotics present.

The probes of this invention are used for analysis of H. pylori within the sample by in situ hybridization, preferably through fluorescence in situ hybridization. The nucleotide sequences of the PNA probes described in this invention are selected from a group with at least 86% structurally similar to the following sequences:

| | |
|---|---|
| 5′- GGG TCT CTC CGT CTT -3′, | (SEQ ID No. 1) |
| 5′- GGG TCT TCC CGT CTT -3′, | (SEQ ID No. 2) |
| 5′- GGG TCT TGC CGT CTT -3′, | (SEQ ID No. 3) |
| 5′- GGG TCT TTC CGT CTT -3′, | (SEQ ID No. 4) |

The development of new PNA-FISH probes is currently carried out empirically. In this particular case, firstly a number of bases that was ideal to the probe application were tested.

A high number of nucleotides within a probe allow a high probe affinity to the target, working at very high temperatures, while a low nucleotide number implies that the binding free energy is not sufficient forhybridization to occur. In this case, the best results were achieved for 12-18 nucleotides.

Additionally, the best sequences for mutation detection were selected. When the pointed out mutations were present in the middle of the sequence of the probes, it was verified that these probes could discriminate between mutations more accurately. However, a diversion of some nucleotides to the left or to the right, only slightly affects the good performance of the probes.

For each particular case, the best experimental FISH conditions was studied, since the probes hybridization success is dependent of hybridization conditions, as well as of fixation/permeabilization and washing steps. Firstly, the conditions referred in a previously published invention (WO2008155742-A2; PT103767-A1), for the detection of Helicobacter pylori were considered. It was predicted that the fixation/permeabilization could be carried out in the same way as earlier describe, i.e., with 4% paraformaldehyde and 50% ethanol, since it was targeting the same microorganism. Therefore, the main studied parameters were temperature, formamide concentration and hybridization and washing times, since these conditions, characteristic from the previous probe can't be extrapolated to this new set of probes.

Because it is necessary to optimize these parameters simultaneously without knowing which one is negatively affecting the PNA-FISH method, this procedure can be complex and time-consuming. It has been referred in studies published by other authors that the low efficiency of some probes turned them helpless for the PNA-FISH procedure. In this case the best time, temperature and formamide concentrations were identified to be 60 minutes of hybridization and 30 minutes of washing step at 55° C. and 50% formamide, or 70° C. and 30% formamide. It is important to refer that due to the fact that temperature and formamide concentration are related is expected that for intermediate formamide concentrations the hybridization temperature may be between 55-70° C.

After a well succeeded hybridization it is possible to assess, by fluorescence microscopy, flow cytometry or real time PCR, the presence/absence, concentration and characterization of a particular microorganism regarding susceptibility to antibiotics.

In this matter it is also important to consider the PNA probe fraction that allows the detection/identification of an existing stable probe/target complex. This detectable fraction of the probe is selected from one of the following groups: a conjugate, a branched detection system, a chromophore, a fluorophore, radioisotope, an enzyme, a hapten or a luminescent compound.

Different probes can be coupled to the same detectable group (e.g. fluorophore) in order to detect H. pylori clarithromycin resistant strains, while another optional probe coupled with a different detectable molecule, can identify, for instance, clarithromycin susceptible strains.

Under the scope of the present invention blocking probes without detectable fraction can also be used in order to reduce or eliminate PNA probe hybridization with non desirable sequences.

The method described on the present invention comprises the contact of a sample with one or more PNA probes described earlier. In accordance with the method the microorganisms within a sample are detected, identified or measured regarding their clarithromycin profile (i.e., susceptible or resistant) correlating the hybridization of the PNA sequence with the target sequence under appropriate hybridization conditions. Consequently the analysis is based in a unique test with a definitive judgment. In contrast the traditional methods currently used for the analysis of microorganisms are based in multiple phenotypic characteristics involving several tests.

It is still the subject of the present invention a kit suitable for carrying out the test to determine, i.e., to detect, identify or measure the presence of H. pylori within samples and/or determine its clarithromycin response (susceptibility/resistance). The kit comprises one or more PNA probes and other selected reagents or compounds needed to undertake in situ hybridization tests.

In a more preferable realization, the kit suitable for performing the H. pylori detection, identification or measurement, comprises a fixation, hybridization and washing solutions.

The method pretends to be a diagnostic, adjuvant mean for a therapeutic disease, leading, to a more adequate treatment in accordance with the test results obtained from a patient sample and from identification of H. pylori strains that are resistant/susceptible to clarithromycin.

The PNA probes can be applied directly on slide samples, since the probes application doesn't involve reagents or enzymes for the permeabilization of the cellular membranes before the hybridization. However, a few compounds that are frequently needed in hybridization are required. Therefore, the probes are normally included in more user-friendly kits.

### DETAILED DESCRIPTION OF THE INVENTION

I—Definitions
a) As used herein, the term “nucleotide” includes natural and artificial molecules known generally by those, who use technology related with nucleic acids, to thereby generate polymers that bind specifically to nucleic acids;
b) When using the term “nucleotide sequence” is the same as referring to a segment of a polymer containing subunits, in this case the nucleotides;
c) The term “target sequence” refers to a nucleotide sequence of Helicobacter pylori that is intended to be detected in the test, where the portion of the nucleotide probe is designed to hybridize;
d) The term “PNA probe” refers to a polymer of subunits of PNA which has a nucleotide sequence and is specific to hybridize with a target sequence of the microorganism of interest. PNA molecules are DNA mimics in which the negatively charged sugar-phosphate backbone structure is replaced by an achiral and electrically neutral formed by repeated N-(2-aminoethyl) glycine units;
e) When using the term “detectable fraction”, it is referred to molecules that can be connected to the probe, to thereby render the probe detectable by an instrument or method;
f) The term “resistant to clarithromycin” in the present invention is related to the analysis of the susceptibility of microorganisms to clarithromycin based on specific genes and point mutations associated with resistance or susceptibility to clarithromycin;
g) The term “sample” refers to any biological sample that may contain the microorganism or target sequence for detection. Preferably, the biological samples are in liquid form (e.g. food, water, blood, urine, etc.) or use as a sample of tissue (e.g. biopsy samples, including gastric biopsies).

II—Description

PNA Probes Conception:

The PNA probes of this invention have as their target sequences of Helicobacter pylori, in areas responsible for mutations related to clarithromycin resistance. Thus, 23S rRNA sequences of various databases were aligned. Three point mutations in position 2142 where an adenine nucleotide is replaced by a cytosine or by a guanine, or in position 2143 were an adenine is replaced by a guanine (Taylor et al., 1997) where located in the peptidyltransferase region encoded in domain V of 23S rRNA gene. These mutations are related with clarithromycin resistance mechanism in this microorganism. An additional probe was designed for the same target location described above, but without any point mutation, allowing the detection of clarithromycin susceptible strains. The PNA probes of this invention comprise 15 nucleotide sequences. In accordance with these criteria, the sequences at least 86% structurally similar to:

| | |
|---|---|
| 5′- GGG TCT CTC CGT CTT -3′; | (SEQ ID No. 1) |
| 5′- GGG TCT TCC CGT CTT -3′; | (SEQ ID No. 2) |
| 5′- GGG TCT TGC CGT CTT -3′; | (SEQ ID No. 3) |
| 5′- GGG TCT TTC CGT CTT -3′ | (SEQ ID No. 4) |

were selected. Despite these probes where described for H. pylori or for their resistances, they are not necessary specific only for this situation.

In alternative, this invention contemplates also variations in probes nucleotide sequences. Such variations can include deletions, insertions among others. Consequently, as it was referred, the probe nucleotide sequence should be at least 86% homologous with the above mentioned sequences.

Detectable Fraction of PNA Probe:

Not limited to the following examples, the detectable fraction of PNA probe can include various types of molecules such as conjugated dextran, chromophores, fluorophores, radioisotopes, enzymes, haptens, chemiluminescent compound, among others.

As an example, among the fluorophores class are preferable to use (but not limited to): Alexa Fluor series, cianines, 5- (and -6) Carboxy-2′,7′-dichlorofluorescein, 5-ROX (5-carboxy-X-rhodamine, triethylammonium salt.

Method:

The present invention discloses a method to H. pylori determination and its clarithromycin resistance. The PNA probes used comprises at least one of the nucleotide sequences at least 86% structurally similar to SEQ ID Nos. 1-4.

The method can contemplate the contact between a sample that have one or more PNA probes described in this document, with the bacteria target sequence under adequate hybridization conditions or adequate in situ hybridization conditions (as seen in example no. 1). Fluorescence in situ hybridization (FISH or PNA-FISH) or real time PCR are the testing formats for H. pylori analysis.

Thus, the method can be divided in: samples preparation, cells fixation, hybridization, washing and results visualization (see example no. 1). The method can be performed in adhered or suspended cells.

Hybridization Conditions:

There are several factors that impose or control stringency of PNA probes hybridization to target sequences. These include the percentage of formamide used (or other chemical denaturant reagent), salt concentration and consequently the ionic strength, temperature of hybridization, the detergent concentration, pH and others. To determine the hybridization optimal conditions it is necessary to fix the different factors and change each factor individually until a desirable discriminatory degree is achieved.

The closer a target sequence is from another non target in the sample, the greater the stringency degree needed to define the various factors that influence the hybridization has to be. In this invention non target sequences can have only one different nucleotide regarding target sequences (since resistance is associated to a difference in a single nucleotide between a susceptible strain and a resistant one), and as such an increased level of discrimination to avoid non specific hybridizations of the PNA probes with non target sequences is necessary. The probe of this invention that hybridizes with H. pylori susceptible strains can be used with the remaining three probes that can detect clarithromycin resistance. However, optionally, the probe can be used without detectable fraction, such as blocking probe, so that, together with optimized hybridization conditions, eliminates, through competition, non-specific binding of the remaining probes to non-target sequences with only one nucleotide of difference (as outlined in example no. 2).

It is generally accepted that one blocking probe acts forming more thermodynamic stable complexes than those formed between the PNA probe and the same sequences, non-target for the PNA probe with detectable fraction), avoiding the latter connection and removing a potential false positive.

Sample Preparation:

The samples to be analyzed can be obtained from biopsies, blood, water, food among others. In biopsies, the samples are cut in 3 to 5 μm slices and placed on slides. In case of H. pylori is in suspension, as in samples of water, air and blood, samples are filtered through a black polycarbonate membrane or equivalent. After that the membranes are placed in slides. For food samples, it is necessary to extract H. pylori from the food matrix, resorting to ultra-sonication or to a stomacher paddle blender after the sample is soaked in water or in a sterile buffer solution. From the moment that H. pylori is in suspension, the applied technique is similar to water, air or blood. Alternatively, H. pylori suspension samples can be submitted directly to the hybridization process.

Kit:

The present invention also refers to a kit that allows testing for H. pylori determination and its clarithromycin resistance.

The PNA probes to use in this kit, its characteristics and the method involved have been previously mentioned herein.

The kit of the present invention comprises at least one nucleotide sequence at least 86% structurally similar to SEQ ID Nos. 1-4, and another reagents or compositions that are selected to perform the test.

The PNA probes, their characteristics, methods and kit of this invention are suitable for the analysis of nucleic acids sequences present or not inside the cells of the organism of interest. As such, this invention can be used for both, analysis of the organism or analysis of nucleic acids extracted or derived from the organism of interest, implying that the source of the target sequence is not a limitation on this invention.

### EXAMPLES

### Example 1

### Detection of Helicobacter Pylori Clarithromycin Susceptible and Resistant Strains

TABLE 1
PNA probes sequences

| | |
|---|---|
| Alexa 488 - O - GGG TCT CTC | Detects Helico- |
| CGT CTT (SEQ. ID No. 1) | bacter pylori |
| Alexa 488 - O - GGG TCT TCC | clarithromycin |
| CGT CTT (SEQ. ID No. 2) | resistant strains |
| Alexa 488 - O - GGG TCT TGC | |
| CGT CTT (SEQ. No. 3) | |
| Alexa 594 - O - GGG TCT TTC | Detects Helico- |
| CGT CTT (SEQ. ID No. 4) | bacter pylori |
| | clarithromycin |
| | susceptible strains |

O = 8-amino-3, 6-dioxaoctanate (makes the connection between the two molecules).
Alexa 488/594 - fluorophore (fraction detectable)

Bacterial Strains:

Several clinical isolates with a clarithromycin resistant status confirmed by PCR were used. Reference strains obtained by American Type Culture Collection, Manassas (ATCC) were also used. A drop of each strain culture was added in 8mm well slides and allowed to dry at 55° C.

Fixation

For preventing the loss of 23S rRNA during the hybridization, the samples were exposed to 4% paraformaldehyde (wt/vol) and 50% ethanol(vol/vol) solutions for 10 minutes each.

Hybridization:

In this step a drop of hybridization solution comprising 10% (wt/vol) dextran sulfate, 10 mM NaCl, 50% (v/v) formamide, 0.1% (wt/vol) sodium pyrophosphate, 0.2% (wt/vol) polyvinylpirroline, 0.2% (wt/vol) FICOLL, 5 mM disodium EDTA, 0.1% (vol/vol) Triton X-100, 50 mM Tris-HCl and 200 nM of PNA probes was added to the sample. The sample was covered with a coverslip to ensure even spreading of the probe and incubated for 60 minutes. During this period the probes could enter the cells membranes and bind to 23S rRNA complementary sequences. The presence of a coverslip and humid paper around samples is essential to prevent the evaporation of the hybridization solution.

Wash:

After hybridization the coverlips were removed and the slides were immersed in a pre-warmed wash solution that consisted of 5 mM Tris Base, 15 mM NaCl and 1% (vol/vol) Triton X-100 (pH 10). The washing step was carried out for 30 minutes at same hybridization temperature.

Results:

The results were obtained trough observation in a fluorescence microscope equipped with filters adapted to the fluorochromes signaling molecules within the probes (i.e., that include the wavelengths of emitting fluorochromes coupled to the probe). It was not detected any signaling when target sequences were absent.

TABLE 2
Results obtained by a fluorescence microscope
equipped with adequate filters for the detection
of the fluorochromes coupled to the probes.

| | Strains | Filter 488 | Filter 594 |
|---|---|---|---|
| Clarithromycin | H. pylori 7.83 | ++ | − |
| resistant | H. pylori 167 | ++ | − |
| strains | H. pylori 6231 | + | − |
| | H. pylori 166 | ++ | − |
| Clarithromycin | H. pylori 968 | − | ++ |
| susceptible | H. pylori NCTC | − | ++ |
| strains | | | |

Filter 488 allows the capture of fluorescence promoted by the fluorochrome Alexa 488, which is indicative of resistance to. clarithromycin. The filter 594 allows the capture of fluorescence promoted by the fluorochrome Alexa 594, which is indicative of susceptibility to clarithromycin.

### Example 2

### Clarithromycin Resistance Detection in Helicobacter pylori Strains Previously Identified

This example aims to illustrate the possibility that, once H. pylori is identified, for example using the method of the document WO2008155742-A2 also published as PT103767-A1, which refers to a PNA probe capable of detecting H. pylori, this invention allows to assess if the strain identified as H. pylori is clarithromycin resistant or susceptible.

TABLE 3
Used PNA probes sequences.

| | |
|---|---|
| Alexa 488 - O - GGG TCT CTC | Detects |
| CGT CTT (SEQ ID No. 1) | clarithromycin |
| Alexa 488 - O - GGG TCT TCC | resistant |
| CGT CTT(SEQ ID No. 2) | Helicobacter |
| Alexa 488 - O - GGG TCT TGC | pylori strains |
| CGT CTT (SEQ ID No. 3) | |
| Alexa 594 -GGG TCT TTC CGT | Blocking probe |
| CTT (SEQ ID No. 4) | |

O = 8-amino-3,6-dioxaoctanate (makes the connection between the two molecules).
Alexa 488/594 - fluorophore (fraction detectable)

Once H. pylori is identified by the previously published method it is possible to study the susceptibility status of the strains. Strains of H. pylori (which were identified using the probe described previously) were tested for clarithromycin resistance using the probes listed in table 3.

It was used the same hybridization protocol from the earlier example, which is different from protocol of the previous cited document WO2008155742 that allows to detect H. pylori. However some changes may be made to enable the use of the probe mentioned above together with the probes of the present invention, allowing the identification and determination of resistance of H. pylori in the same test.

Results:

The results were obtained trough observation in a fluorescence microscope equipped with filters adapted to the fluorochromes signaling molecules within the probes.

TABLE 4
Filter 488 allows the capture of fluorescence
promoted by the fluorochrome Alexa 488, which
is indicative of resistance to Clarithromycin.

| | Strains | Filter 488 |
|---|---|---|
| Clarithromycin | H. pylori 6.236 | + |
| resistant | H. pylori 7.002 | ++ |
| strains | H. pylori 168 | ++ |
| | H. pylori 7.11 | ++ |
| Clarithromycin | H. pylori 169 | − |
| susceptible | H. pylori NCTC | − |
| strains | | |

1. PNA probe for detecting the presence of Helicobacter pylori strains, or detection of Helicobacter pylori response to clarithromycin, or detection of Helicobacter pylori and its response to clarithromycin, which comprises at least one of the nucleotide sequences at least 86% structurally similar to SEQ ID Nos. 1-4.

2. PNA probe, in accordance with claim 1, capable of detecting the target sequence in rRNA, rDNA or in Helicobacter pylori complementary rRNA sequences.

3. PNA probe, in accordance with claim 1, capable of detecting clarithromycin resistance when comprising at least one of nucleotide sequences at least 86% structurally similar to SEQ ID Nos. 1-3.

4. PNA probe, in accordance with claim 1, capable of detecting clarithromycin susceptibility when comprising at least one of nucleotide sequences at least 86% structurally similar to SEQ ID Nos. 4.

5. PNA probe, in accordance with claim 1, wherein the probe is connected at least to one detectable fraction.

6. PNA probe, in accordance with claim 5, wherein the type of detectable fraction of the probe is selected from the following groups: a conjugate, a branched detection system, a chromophore, a fluorophore, radioisotope, an enzyme, a hapten or a luminescent compound.

7. PNA probe, in accordance with claim 6, wherein the fluorophore group is at least one of the following: fluorophores Alexa series, Alexa Fluor series, cyanine, 5- (and -6) Carboxy-2′,7′-dichlorofluorescein , the 5-ROX (5-carboxy-X-rhodamine, triethylammonium salt).

8. Kit for detecting the presence of Helicobacter pylori strains, or detection of Helicobacter pylori response to clarithromycin, or detection of Helicobacter pylori and its response of Helicobacter pylori to clarithromycin in biological samples comprising at least one of the probes described in claim 1.

9. Kit, according to claim 8, comprising at least one of the following solutions: one fixation solution, one hybridization solution and one washing solution.

10. Kit, according to claim 8, wherein the fixation solution comprises 2-8% (wt/vol) of paraformaldehyde and 25-75% (vol/vol) of ethanol.

11. Kit, according to claim 9, wherein the hybridization solution comprises formamide.

12. A method for detection of Helicobacter pylori, or detection of H. pylori clarithromycin response or detection of H. pylori and response of Helicobacter pylori to clarithromycin in biological samples, comprising the use of at least one of the PNA probes described in claim 1 for comprising the following steps:
a. PNA probe contacting the referred samples;
b. PNA probe hybridizing the target sequence of microorganisms present in the referred samples;
c. Hybridization detecting as indicative of the referred detection and measuring of the referred samples.

13. Method, according to claim 10, wherein the biological sample is derived from blood, air, food, water, biopsies or gastric biopsies.

14. Method, according to claim 12, wherein the hybridization occurred by fluorescence.

15. A method of using PNA probes of claim 1, comprising applying in a methodology for detecting Helicobacter pylori, or in detection of Helicobacter pylori clarithromycin response, or detection of Helicobacter pylori and response of Helicobacter pylori to clarithromycin in biological samples.

16. A method of using the kit of claim 8, to be applied on detecting Helicobacter pylori, or in detection of Helicobacter pylori clarithromycin response, or detection of Helicobacter pylori and response of Helicobacter pylori to clarithromycin in biological samples.

17. The method according to claim 12, to be applied on detecting Helicobacter pylori, or in detection of Helicobacter pylori clarithromycin response, or detection of Helicobacter pylori and response of Helicobacter pylori to clarithromycin in biological samples.


SEQUENCE LISTING (4 sequences)

SEQ ID NO: 1 - DNA, 15 bp, Helicobacter pylori
gggtctctccgtctt

SEQ ID NO: 2 - DNA, 15 bp, Helicobacter pylori
gggtcttcccgtctt

SEQ ID NO: 3 - DNA, 15 bp, Helicobacter pylori
gggtcttgccgtctt

SEQ ID NO: 4 - DNA, 15 bp, Helicobacter pylori
gggtctttccgtctt